(19) United States
(12) Patent Application Publication
(10) Pub. No.: US 2014/0074056 A1
(43) Pub. Date: Mar. 13, 2014

(54) Means and apparatus for controlling oral fluids during sleep

(71) Applicant: Anna Bracero, Springhill, FL (US)

(72) Inventor: Anna Bracero, Springhill, FL (US)

(21) Appl. No.: 13/573,401

(22) Filed: Sep. 12, 2012

(51) Int. Cl. A61F 13/58

(52) U.S. Cl. CPC A61F 13/58
USPC 604/385.03

(57) ABSTRACT

The apparatus comprises an absorbent material adjacent to an adhesive material which is further adapted to protect and prevent saliva or other oral fluids from being released to soil bedding material or to cause discomfort during sleep.

### FIELD OF THE INVENTION

The invention relates to personal hygiene items useful in controlling the flow of will be, almost immediately upon, such as drool, during times of sleep.

### BACKGROUND OF THE INVENTION

During times of sleep it is not uncommon for individuals to exhibit behavior and habits which are inconvenient or uncomfortable either for themselves or for a sleeping partner. While snoring is one common problem, it is also true that the secretion and expulsion of oral fluids, such as drool and spittle, can result in interruption of sleep, staining or soiling of bed material, or discomfort to a sleeping partner.

For this reason it would be useful to find some means of controlling the flow of such oral fluids. There have been a number of efforts made to capture and dispose of such fluids, but most of them include either a substantial and uncomfortable apparatus or they rely upon the controlled target fluid to flow through or dropped from the face before it is captured.

For instance, the patent issued to Brown, et al, U.S. Pat. No. 5,980,498, issued on Nov. 9, 1999, discloses what is described as a “portable saliva compensation device”. It is an apparatus designed to fit about the midsection of a person and includes a trough (or lack of a better term) which is further adapted with a two and a storage area in the rear. It is adapted to capture saliva which may drip of a person and gather it in the rear or disposal. This apparatus clearly would only be useful for a person in the upright position and requires a separate action to dispose of the saliva. This apparatus was enhanced with additional electrical circuitry and U.S. Pat. No. 6,652,481, issued on Nov. 25, 2003. The purpose of this invention is twofold. First to capture the saliva and second to provide means of measuring the effectiveness of the device.

Also of note is U.S. Pat. No. 7,269,856, issue to Cannan on Sep. 18, 2007, entitled “Dribble Bib”. This apparatus is simply an improved form of a bib which would seem to be primarily intended for use by infants while eating in an upright position for by others who may have difficulty managing eating functions.

What is lacking is some concern for undesirable flow of fluids during times of sleep. Such an apparatus would need to be adapted for use by a person in other than an upright position and would also need to be unobtrusive so as to avoid disturbing a good night's sleep. It would also be desirable for such a device to absorb the fluids overnight for disposal at a convenient time.

What is not provided but would be useful is a simple and unobtrusive device which would be useful in controlling a substantial amount of the drool or fluid dripping from or emanating from a person's mouth during times of sleep.

### SUMMARY OF THE INVENTION

The inventor has developed an apparatus and means for achieving this goal. In short, the apparatus comprises an adhesive bandage which is further adopted to enclose a blotting material and is shaped so as to fit in proximity to a person's mouth during times of sleep so that such a bodily fluids will be, almost immediately upon emanation from the mouth, plotted and absorbed into a material which will store the fluid or moisture until the sleeping party is naturally and rest only awakened.

The apparatus may be manufactured in certain standard shapes and designs by sizes or it may be manufactured in a way to permit it to be shaped for placement in a particular manner to accommodate and individuals sleeping patterns or habits. Such could further be accomplished by manufacturing the basic configuration of materials in sheets which could be cut as needed to fit a particular person.

The apparatus may also be made of a variety of materials. Certain materials might be more absorbent while other materials might sacrifice some absorbency for a measure of comfort. For the same reasons, certain adhesive materials may be more harsh than others while others may sacrifice some degree of adhesion for a bit more comfort both during sleep and as the apparatus is removed.

It is, then, an object of the invention to provide an unobtrusive and simple means of diverging oral fluids, such as drool or spittle, from interfering with the comfort and quality of sleep by either a sleeping person or a sleeping partner.

It is another object of the present invention to provide such and unobtrusive and simple means of blotting for absorbing such oral fluids during times of sleep so that they will not cause discomfort to a sleeping party or sleeping partner or stain or soil the bed material, such as a pillow, of such sleeping person or sleeping partner.

It is a further object of the present invention to provide such an unobtrusive and simple means of controlling the flow of such oral fluids during times of sleep with standard sizes and shapes of blotting material which may be properly positioned by use of an adhesive

It is a further object of the present invention to provide such an unobtrusive and simple means of controlling the flow of such oral fluids during times of sleep with a materials which is both adhesive and has blotting properties in which may be easily shaped and positioned by an individual to achieve the maximum control with the minimum interference or interruption with standard sleeping positions of a sleeping person or a sleeping partner.

It is a further object of the present invention to provide such a simple and unobtrusive means which may be made of a variety of absorbent materials tailored to meet the appropriate degrees of comfort and absorbency.

Other features and advantages of the present invention will be apparent from the following description in which the preferred embodiments have been set forth in conjunction with the accompanying drawings.

### BRIEF DESCRIPTION OF THE DRAWINGS

In describing the preferred embodiments of the invention reference will be made to the series of figures and drawings briefly described below.

FIG. 1 depicts such an adhesive blotting apparatus made in a standard shape or configuration so as to fit about a person's lower left side of their mouth.

FIG. 2 depicts an adhesive blotting apparatus positioned on a sleeping person so as to capture drool or spittle which may be involuntarily expelled from a person's mouth during times of sleep.

FIG. 3 depicts such an adhesive blotting apparatus made in a desired shape which may be positioned so as to fit or satisfy the needs of a particular sleeping person.

FIG. 4 depicts a cross-section of blotting apparatus which further depicts how the blotting material may be encased between an adhesive piece and an enclosing surface.

FIG. 5 depicts a sheet of blotting apparatus material which may be cut into individual shapes for use as desired.

While certain drawings have been provided in order to teach the principles and operation of the present invention, it should be understood that, in the detailed description which follows, reference may be made to components or apparatus which are not included in the drawings. Such components and apparatus should be considered as part of the description, even if not included in such a drawing. Likewise, the drawings may include an element, structure, or mechanism which is not described in the textual description of the invention which follows. The invention and description should also be understood to include such a mechanism, component, or element which is depicted in the drawing but not specifically described.

### DETAILED DESCRIPTION OF THE PREFERRED EMBODIMENT

Reference will now be made in detail to the present preferred embodiment of the invention, an example of which is illustrated in the accompanying drawings. While the invention will be described in connection with a preferred embodiment, it will be understood that it is not intended to limit the invention to that embodiment. On the contrary, it is intended to cover all alternatives, modifications, and equivalents as may be included within the spirit and scope of the invention defined in the appended claims.

While the following description will seek to improve understanding of the invention by describing the various components and elements, it should be considered that certain apparatus may be sufficiently and adequately explained by the accompanying drawings, which are fully incorporated herein, and not require further description. All such apparatus should be considered as part of the specification of the invention for all purposes.

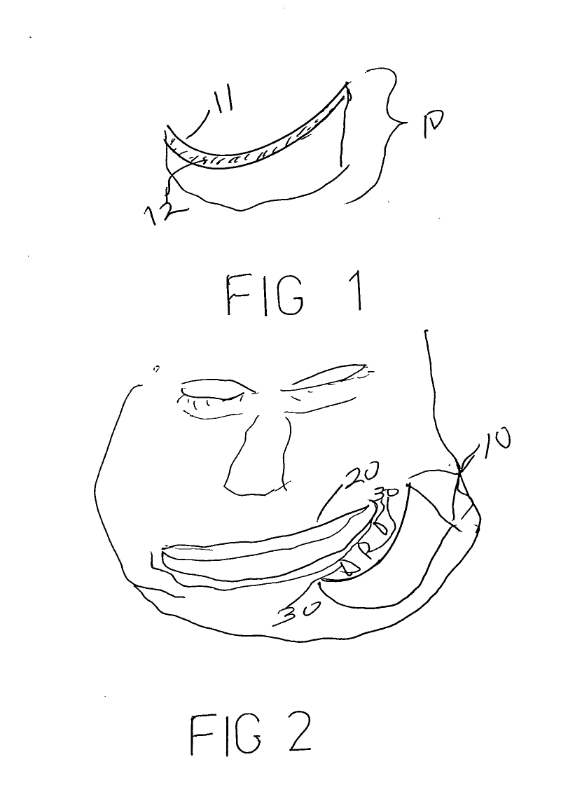

Making reference first in FIG. 1 it can be seen that the adhesive blotting apparatus according to the present invention (10) comprises an adhesive (11) so that drool or spittle which may be expelled from a person's mouth during sleep will flow down to the blotting apparatus (12) so that it may be captured and absorbed by a blotting material.

Making reference now to FIG. 2 it can be seen that the device, once positioned properly in the vicinity of a person's mouth (20), will be able to receive whatever fluids. Such as drool or spittle (30) that may pass from a person's mouth (20) during sleeping hours. It is assumed, for the purpose of this invention, that most persons will sleep on their back or side. If a person was sleeping on their stomach with their face down, it is likely and probable that such escaping fluids would be absorbed by the bedding material and this apparatus would not be useful.

It can be seen that it be that such will indeed be in a proper position to the fluids and will stay in place such as to be capable of absorbing and maintaining a reasonable amount of such rule fluid unless such amounts to a flow which would be indicative of a more serious medical issue. It should be noted that the adhesive may be selected from the variety of adhesive materials which are adapted to be placed upon a person to capture and then remove a minor amount of drool or spittle with as little discomfort as possible.

A few comments should be made about the materials selected for use with the apparatus. There are a variety of adhesives for the skin. These range from the type used for simple Band-Aids to the type used for more heavy-duty adhesions, such as to hold gauze strips in place. The apparatus could be made with any of these adhesives and would be selected by each person so as to provide the minimal amount of adhesion to keep the apparatus in place overnight while minimizing the discomfort associated with the overnight adhesion to the face as well as the removal in the morning.

Similarly, the blotting material can be selected from a variety of materials ranging from cloth to synthetic material and should be selected so as to reliably block and contain the amount of fluid associated with each individual person. Some people may release considerably more drool and spittle than others and this should be taken into consideration when selecting each version of this apparatus.

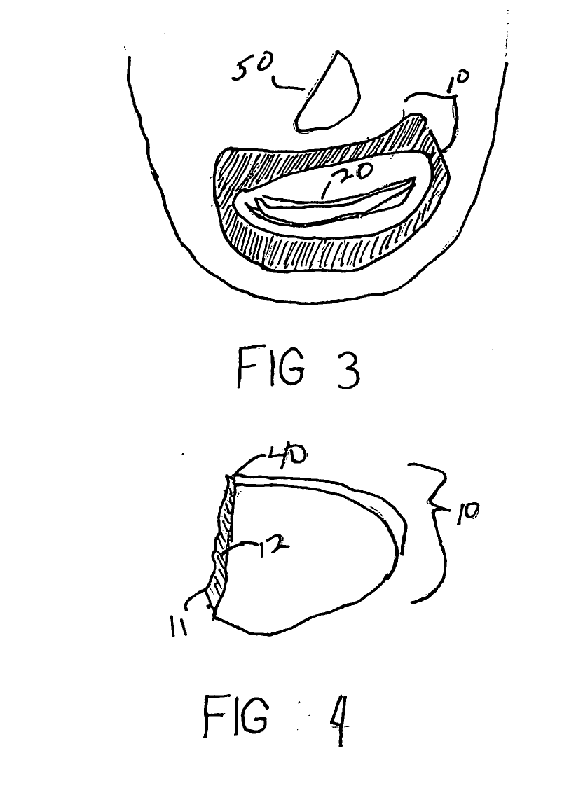

Making reference now to FIG. 3, it can be seen that the apparatus could be manufactured of a desired shape to fit completely around the mouth and under the nose. This would accommodate a person who released fluid from both the nose and the mouth during the night. Of course, such a shape could also be sized so as to fit persons of various sizes and physiques. The variety of sizes and shapes are endless depending upon the needs and size of each individual person. The apparatus could be manufactured in these various sizes and shapes as desired.

Making reference now to FIG. 4, it can also be seen that the apparatus could also be made with a covering sheet (40) so as to in close or completely capture the fluid released by the nose or mouth. This would be an alternate embodiment of the basic invention which is keeping in the spirit and scope of the original invention.

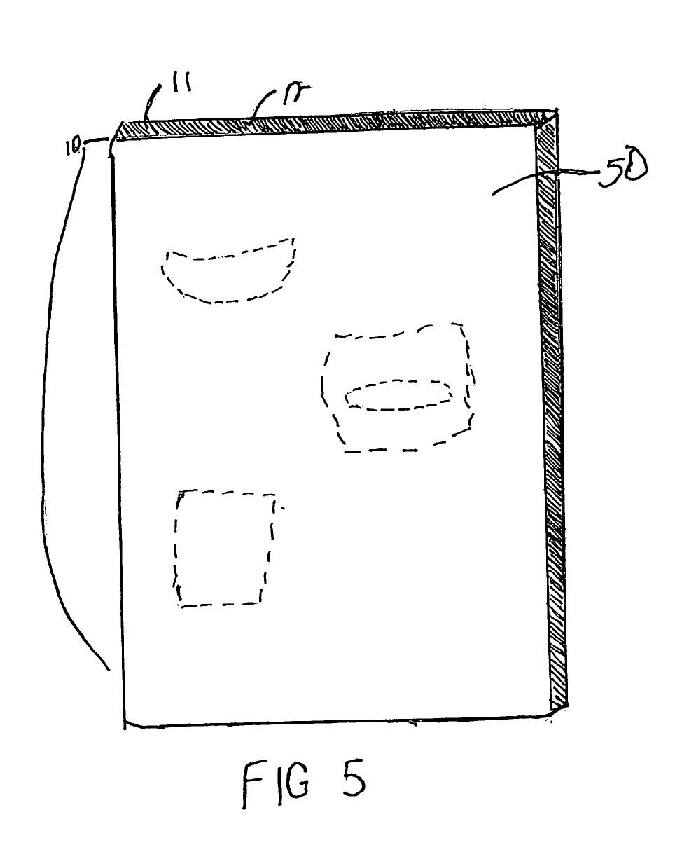

Finally, making reference to FIG. 5, it can further be seen that the basic apparatus could be manufactured in a sheet (50) so that each individual consumer could fashion and apparatus tailored to their individual needs by simply cutting a desired size and shape from the sheet with a scissors.

Because of the abdomen and start over that area because of the way these of the materials selected and because such materials will be placed upon a person's skin and used to absorb bodily fluids is not anticipated that such apparatus would ever be reused. Accordingly, the these materials together with the simple construction and design make it possible for a person to fly a new one every night and then discard attributes area of those were in such link and expiration of fluids only is a problem because would be well worthwhile. Allowing easy recovery and that he needs the material could be adapted with a backing to protect it from being compromised during shipment and storage for sale

The apparatus, as described herein, is capable of several modifications, some of which have been already discussed. For instance, the items could be manufactured according to set sizes and a sufficient variety to ensure appropriate fit for each person. On the other hand, the items could be manufactured so as to permit minor modification by the user, for instance having or slight reshaping, to permit mass manufacture of a standard size which could be modified or adjusted for each use by the concerned person or to achieve not only some measure of cost savings but also to improve the comfort and efficiency.

The basic apparatus has been described with particular variations with respect to the configuration, size, shape, materials, and thicknesses of materials to be used. Each of these variations should be seen as keeping in the spirit and scope of the present invention. It should also be seen that each of variations may be used with other variations or alone. They are not to be seen as exclusive but as inclusive.

Further modification and variation can be made to the disclosed embodiments without departing from the subject and spirit of the invention as defined in the following claims. All such modifications and variations, as included within the scope of these claims, are meant to be considered part of the invention as described.

What is claimed is:

1. Apparatus for capturing and overnight storage of oral and nasal fluids released by a person during time of sleep, said apparatus further comprising:
an adhesive material which is adapted to adhere to a person's skin in areas proximate to a person's nose and mouth during time of sleep; and
an absorbent material which is adapted to absorb and overnight retain fluids which may be released from a person's nose or mouth during time of sleep.

2. Apparatus for capturing and overnight storage of oral and nasal fluids released by a person during time of sleep, said apparatus further comprising:
a sheet which further comprises an adhesive side which further comprises an adhesive material which is adapted to adhere to a person's skin;
an absorbent side, which further comprises a material which is adapted to absorb and overnight retain fluids which may be released from a person's nose or mouth during time of sleep; and
said sheet further being adapted to allow cutting in patterns so that a person may select a size and shape appropriate for such person space to facilitate thorough and comfortable positioning for the capture and overnight retention of fluids which may be released from said person during time of sleep.

3. Apparatus for capturing and overnight storage of oral and nasal fluids released by a person during time of sleep, said apparatus further comprising:
an adhesive material which is adapted to adhere to a person's skin in areas proximate to a person's nose and mouth during time of sleep;
an absorbent material which is adapted to absorb and overnight retain fluids which may be released from a person's nose or mouth during time of sleep; and
a covering surface on the opposite side of said absorbent material from the adhesive material which is adapted to facilitate the capture of a relatively large amount of said oral or nasal fluid which may be released from said person during time of sleep.

4. Apparatus for capturing and overnight storage of oral and nasal fluids released by a person during time of sleep as described in claim 1, in which said adhesive material may be selected from a range of adhesive materials which may provide only minimal adhesion to one which may provide a stronger adhesion so long as said material is one which is safe and sanitary for use on a person's skin.

5. Apparatus for capturing and overnight storage of oral and nasal fluids released by a person during time of sleep as described in claim 1, in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

6. Apparatus for capturing and overnight storage of oral and nasal fluids released by a person during time of sleep as described in claim 1, in which said adhesive material may be selected from a range of adhesive materials which may provide only minimal adhesion to one which may provide a stronger adhesion so long as said material is one which is safe and sanitary for use on a person's skin, and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

7. Apparatus for capturing and overnight storage of oral and nasal fluids released by a person during time of sleep as described in claim 1, in which said apparatus may be made of a desired size and shape to fit the area proximate to a persons mouth and nose to satisfy the individual needs of said person.

8. Apparatus for capturing and overnight storage of oral and nasal fluids released by a person during time of sleep as described in claim 1, in which said apparatus may be made of a desired size and shape to fit the area proximate to a persons mouth and nose to satisfy the individual needs of said person and in which said adhesive material may be selected from a range of adhesive materials which may provide only minimal adhesion to one which may provide a stronger adhesion so long as said material is one which is safe and sanitary for use on a person's skin.

9. Apparatus for capturing and overnight storage of oral and nasal fluids released by a person during time of sleep as described in claim 1, in which said apparatus may be made of a desired size and shape to fit the area proximate to a persons mouth and nose to satisfy the individual needs of said person and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

10. Apparatus for capturing and overnight storage of oral and nasal fluids released by a person during time of sleep as described in claim 1, in which said apparatus may be made of a desired size and shape to fit the area proximate to a persons mouth and nose to satisfy the individual needs of said person and in which said adhesive material may be selected from a range of adhesive materials which may provide only minimal adhesion to one which may provide a stronger adhesion so long as said material is one which is safe and sanitary for use on a person's skin, and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

11. Apparatus for capturing and overnight storage of oral and nasal fluids released by a person during time of sleep as described in claim 2, in which said adhesive material may be selected from a range of adhesive materials which may provide only minimal adhesion to one which may provide a stronger adhesion so long as said material is one which is safe and sanitary for use on a person's skin.

12. Apparatus for capturing and overnight storage of oral and nasal fluids released by a person during time of sleep as described in claim 2, in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

13. Apparatus for capturing and overnight storage of oral and nasal fluids released by a person during time of sleep as described in claim 2, in which said adhesive material may be selected from a range of adhesive materials which may provide only minimal adhesion to one which may provide a stronger adhesion so long as said material is one which is safe and sanitary for use on a person's skin, and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

14. Apparatus for capturing and overnight storage of oral and nasal fluids released by a person during time of sleep as described in claim 2, in which said apparatus may be made of a desired size and shape to fit the area proximate to a persons mouth and nose to satisfy the individual needs of said person.

15. Apparatus for capturing and overnight storage of oral and nasal fluids released by a person during time of sleep as described in claim 2, in which said apparatus may be made of a desired size and shape to fit the area proximate to a persons mouth and nose to satisfy the individual needs of said person and in which said adhesive material may be selected from a range of adhesive materials which may provide only minimal adhesion to one which may provide a stronger adhesion so long as said material is one which is safe and sanitary for use on a person's skin.

16. Apparatus for capturing and overnight storage of oral and nasal fluids released by a person during time of sleep as described in claim 2, in which said apparatus may be made of a desired size and shape to fit the area proximate to a persons mouth and nose to satisfy the individual needs of said person and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

17. Apparatus for capturing and overnight storage of oral and nasal fluids released by a person during time of sleep as described in claim 2, in which said apparatus may be made of a desired size and shape to fit the area proximate to a persons mouth and nose to satisfy the individual needs of said person and in which said adhesive material may be selected from a range of adhesive materials which may provide only minimal adhesion to one which may provide a stronger adhesion so long as said material is one which is safe and sanitary for use on a person's skin, and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

18. Apparatus for capturing and overnight storage of oral and nasal fluids released by a person during time of sleep as described in claim 3, in which said adhesive material may be selected from a range of adhesive materials which may provide only minimal adhesion to one which may provide a stronger adhesion so long as said material is one which is safe and sanitary for use on a person's skin.

19. Apparatus for capturing and overnight storage of oral and nasal fluids released by a person during time of sleep as described in claim 3, in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.

20. Apparatus for capturing and overnight storage of oral and nasal fluids released by a person during time of sleep as described in claim 3, in which said adhesive material may be selected from a range of adhesive materials which may provide only minimal adhesion to one which may provide a stronger adhesion so long as said material is one which is safe and sanitary for use on a person's skin, and in which said absorbent material may be selected from a range of absorbent materials which further may provide a range of absorbency from minimal necessary for absorption of a persons oral and nasal fluids which provides only minimal absorption to one which may provide the absorption necessary to absorb and overnight retain the oral and nasal fluids from a person which may release a relatively heavy amount of oral or nasal fluids.
